(19) United States
(12) Patent Application Publication
(10) Pub. No.: US 2011/0008797 A1
(43) Pub. Date: Jan. 13, 2011

(54) METHOD AND DEVICE FOR THE THERMAL CONTROL OF TEMPERATURE-DEPENDENT ENZYMATIC REACTIONS USING MAGNETIC PARTICLES OR MAGNETIC BEADS AND ALTERNATING MAGNETIC FIELDS

(75) Inventors: Christian Zilch, Leipzig (DE); Wilhelm Gerdes, Leipzig (DE); Johann Bauer, Darmstadt (DE); Karl Holschuh, Seeheim-Jugenheim (DE)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignees: Fraunhofer-Gesellschaft zur Foederung der angewandten Forschung e.V., Muenchen (DE); Merck Patent GmbH, Darmstadt (DE)

(21) Appl. No.: 12/922,470

(22) PCT Filed: Mar. 19, 2009
(86) PCT No.: PCT/EP09/02052

§ 371 Date: Sep. 14, 2010

(30) Foreign Application Priority Data
Mar. 20, 2008 (DE) 08005370.5

(51) Int. Cl. C12Q 1/68; C12P 19/34; C12M 1/00; C12M 1/34

(52) U.S. Cl. 435/6; 435/91.2; 435/289.1; 435/287.1; 977/902

(57) ABSTRACT

The invention relates to a method for the thermal control of at least one temperature-dependent enzymatic reaction in the presence of magnetic particles, particularly nanoparticles, or magnetic beads in vitro by means of the heating of the magnetic beads or magnetic particles to at least one certain target temperature by means of alternating magnetic fields. The enzymatic reaction which can be controlled with the method according to the invention is preferably a PCR reaction or another reaction for the elongation or amplification of nucleic acids, including DNA, RNA or hybrids or derivatives thereof, which takes place directly on the functionalized magnetic beads. Further aspects of the invention relate to a reactor for carrying out the method and the use of the method or the reactor in analytics and diagnostics.

Analytical and diagnostic methods, which are in particular used for the processing of a sample, the isolation of target structures or molecules, the amplification of target molecules and the detection of target molecules, have a broad and constantly growing spectrum of fields of application. With the advancements made in the fields of genetic engineering and molecular biology, methods in which biological and chemical reactions with nucleic acids as reactants or target molecules play an important role in particular have recently increasingly gained in importance.

Various amplification methods are used for the amplification and detection of specific sequences in nucleic acids (RNA, DNA). The most common method is based on the polymerase chain reaction (PCR) with which a specific nucleic acid sequence within a DNA strand (template) can be amplified. PCR can be used as part of a nucleic acid assay or as a stand alone method. Here, with the aid of a DNA polymerase free deoxynucleotides are added to oligonucleotide sequences (primers) hybridized to a template. The resulting amplification product can then be thermally split again and used as a template for further reactions. In this manner, a target sequence, which is present at a low concentration in a DNA sample, can be amplified exponentially.

To carry out a PCR, short sense and antisense oligonucleotides, which delimit the region to be amplified, are added to a reaction mixture with the template in a low concentration, a thermostable polymerase and individual nucleotides. Generally, the reaction mixture is subjected to a plurality of temperature cycles (denaturing: approx. 95-100° C., hybridization: 40-65° C., elongation: 70-80° C., preferably approx. 72° C. depending on the polymerase used) in a closed vessel (capillary or tube) suitable for that purpose. The temperature cycles are generally generated with a peltier element as thermoblock or hot air (LightCycler). To this end, a direct contact between vessel wall and peltier element or alternatively capillary wall and circulating hot air must take place. The achievement of an efficient heat transmission between the PCR reaction mixture and the heating medium, (peltier element, hot air) is more frequently problematic, so that the temperature gradients cannot be realized optimally. Particularly in the case of automated processes, such as fully integrated diagnostics systems, which require an amplification step for example by means of PCR, the direct coupling of the peltier element or a hot air nozzle to a disposable (reagent cartridge) is further required. To this end, it generally requires a high mechanical outlay.

It would therefore be very advantageous if the temperature in the case of such nucleic acid amplification reactions, such as e.g. the required temperature cycles of the PCR, could be controlled in a simple and effective manner contactlessly. Although fundamentally there is a range of known methods for the contactless temperature control of chemical and biological reactions, e.g. the use of microwaves, infra-red light, laser irradiation, halogen light, hot air, induction heating, chemical heating methods, etc. (see e.g. Biotechnology Advances 24 (2006), pp. 243-284), most of these methods entail a relatively high outlay in terms of apparatus and/or are connected with other disadvantages. The temperature control of these methods in particular is relatively imprecise, so that the use of temperature sensors is necessary for exact setting, which temperature sensors must be placed into the reaction space or in direct contact with the reaction space.

The present invention is based on the surprising observation that magnetic beads in the reaction volume can be heated very precisely and quickly to certain target temperatures by means of magnetic alternating fields and thus a thermal control of reactions for the amplification or elongation of nucleic acids, PCR in particular, can be achieved in a convenient and efficient manner without conventional heating elements or heating media being necessary. In the case of precise presetting of the heating conditions by means of the type and quantity of the magnetic particles/beads, the use of temperature sensors can in many cases also be dispensed with.

The combination of nucleic acids with magnetic beads is known in principle.

For some time, magnetic particles (nanoparticles and beads) have been used for the purification of macromolecules such as nucleic acids and proteins, but also viruses, bacteria and mammalian cells. To this end, capture molecules (e.g. antibodies, haptens, nucleic acids) targeted e.g. at certain specific surface structures on the biomolecules are immobilized on the surface of the magnetic beads. Alternatively, the surface of the magnetic beads can be equipped with special coatings, such as e.g. silanes, which have a high affinity for a molecule sort. The absorption of the target molecules then takes place under specific buffer conditions. By means of a change of the buffer medium, the molecules bound to the surface can be detached again (eluted).

The separation of the target structure or of the target molecule from the remainder of the sample and its subsequent purification takes place by means of the application of a magnetic field which concentrates and reversibly fixes the magnetic beads out of the suspension and in one region. The supernatant can then be disposed of and for example be replaced by a washing buffer in order to purify the target molecules bound to the beads. The beads are re-suspended by means of the removal of the outer magnetic field.

For the most part, in nucleic acid based purification methods, silanized magnetic beads which have a high binding affinity for the positively charged backbone of nucleic acids (DNA, RNA) are used. In the case of the use of a high concentration of so-called chaotropic salts, which reduce a hydrophilic interaction with ions, in the buffer solution, nucleic acids bind reversibly to the bead surface. This bond is dissolved again by means of a reduction of the salt concentration or temperature increase.

Recently, also single-stranded nucleic acids (oligonucleotides, cDNA or PCR products) were bound by means of linker molecules onto the bead surface, in order to enable a specific hybridization with complementary nucleic acid strands present in a sample.

Recently, approaches have been described, in which an elongation of oligonucleotides, which are present directly or coupled by means of spacers on magnetic bead carriers, is possible. Liu et al. (2001) describe a PCR using primers which are bound to magnetic nanoparticles, wherein the PCR products are amplified directly on the nanoparticles (Biotechnol. J. 2007 Apr. 2(4):508-11).

Bead-bound primers are also used inter alia in high-throughput screening methods. So, this method is used for example by Kojima et al. (2005) for the screening of transcription factors using PCR amplification (Nucleic Acids Research, 2005, Vol. 33, No. 17).

Furthermore, it is known that the application of external alternating magnetic fields within certain frequencies brings about a heating of magnetic particles in suspensions. This effect is applied in particular in “magnetic particle hyperthermia” in the case of tumor diseases. After injecting the magnetic particles into the tumor tissue, a localized heating and destruction of the tumor tissue arises following the switching on of alternating magnetic fields by means of an external electromagnet (U. Gneveckow, A. Jordan, R. Scholz, et al., Biomed. Technol. 50 (2005) 92; and M. Suzuki, M. Shinkai, M. Kamihira et al., Biotechnol. Appl. Biochem. 21 (1995) 1179). This effect can also be exploited in “drug targeting” for targeted control in vivo and local release of therapeutically active substances (E. Viroonchatapan, H. Sato, M. Ueno et al., Life Sci. (1996) 58(24):2251-61).

Porous magnetic particles, in the pores of which associated material can be embedded, are described in WO 2008/027090 A2. The particles and the associated material can be heated by means of electromagnetic fields and their binding properties and/or other properties can be controlled by means of the heating.

DE 198 00 294 A1 describes magnetic polymer particles which can be heated up inductively and also methods for their production and functionalization with various affinity ligands, and, in a general form, various use options, particularly also in biomedical analytics.

WO 2004/052527 A1 discloses a cyclic flow reactor for a PCR reaction, in which the heating takes place in a manner imparted by beads via high-frequency magnetic fields.

However, no use has hitherto been found for hyperthermia in the control of nucleic acid amplifications or primer elongations which are carried out directly on (primer)-functionalized beads, e.g. of PCR on the bead and similar reactions.

The object of the present invention was therefore the provision of a new method for the contactless thermal control of temperature-dependent enzymatic reactions in vitro, which is suitable in particular for the control of nucleic acid amplification reactions, such as e.g. PCR or nucleic acid elongation reactions “on the bead” and requires a lower outlay in terms of apparatus.

This object is achieved according to the invention by means of the provision of the method according to claim 1 and also of the reactor according to claim 14. Further aspects and/or preferred embodiments of the invention are the subject of the further claims.

In the method according to the invention, the relevant reactions are thermally controlled contactlessly in vitro by means of the heating of magnetic particles or beads in the reaction space to at least one target temperature with the aid of alternating magnetic fields.

In a preferred embodiment of the invention, the temperature cycles of a PCR on a bead are generated by means of external alternating magnetic fields which lead to a heating of the particles. To this end, the magnetic beads are designed in such a manner that these are thermally stable, oligonucleotides are able to bind to their surface stably (covalently) and can be excited by means of external alternating magnetic fields in such a manner that a heating (hyperthermia) takes place. The heat generation is induced in dependence on the magnetic properties and size of the beads by means of an alternating magnetic field at a frequency which preferably lies in the 50-500 kHz frequency band.

The heating up of the magnetic particles or beads for the temperature control of a PCR or another reaction can be calibrated by means of the Curie temperature of the alloys used for the nanoparticles, the particle content per bead and the number of beads per (PCR) reaction volume. The heating of the reaction mixture takes place via external alternating magnetic fields of defined field strength and frequency, in order to generate optimal temperature gradients. The cooling of the reaction mixture takes place passively or actively by means of air cooling or coupling to a thermally conductive material. The heating up and subsequent cooling is based on the so-called magnetocaloric effect which is meanwhile used in magnetic heat pumps and refrigerators.

Suitable field strengths typically lie in a range of 1 to 1000 kA/m, preferably of 1 to 200 kA/m, e.g. 1 to 20 kA/m or, for higher heating outputs, 10 to 100 kA/m, more preferably to 100 kA/m, and depend on whether ferro, para- or superparamagnetic particles are used and predominantly the heating effect caused by means of the Brown or the Neel mechanism becomes effective. Suitable frequencies typically lie in a range of 1-1000 kHz, preferably 50-500 kHz and more preferably in a range of 100-400 kHz. For the generation of the alternating magnetic field, e.g. a field strength of 1.6 kA/m in the kHz range, e.g. 300 kHz, is suitable, with which a heating output of approximately 5 kW can be achieved in the field.

The field strengths and frequencies can readily be optimized for special use case by means of routine experiments by the person skilled in the art.

The temperature increase which can be achieved by means of magnetic beads or magnetic particles in a reaction volume results from the power loss Qs in the electromagnetic field which a certain quantity of magnetic particles transfers to the surrounding medium (e.g. water or an aqueous solution). This calculation requires that the power loss in the alternating field is practically completely converted into heat (confirmed in practice by empirical results).

Under the typical above field strength and frequency conditions, the specific power loss for particles of 13 nm to 100 nm (determined by means of BET nitrogen adsorption) generally lies in the range of approximately 100 to 600 W/g or kJ/kg s.

This power loss can be determined empirically for various beads or magnetic particles without difficulty and particles which are suitable with reference to size and composition for a desired heat output can be selected.

The following equation applies for magnetite particles (Fe₃O₄):
Heating rate ΔT=Qs m (Fe₃O₄)/c(H₂O)×m(H₂O)
Heat capacity c(H₂O)=4.18 kJ/kg K
Specific power loss of particles with 25 nm diameter:
approx. 250 kJ/kg s

A typical reaction batch for a PCR or another enzymatic reaction involving nucleic acids comprises a volume of approximately 5 μl to 1 ml, more often 10 μl to 500 μl, typically 50 μl to 100 μl. In the case of a volume of 50 to 100 μl (0.05 g to 0.1 g) and a particle quantity of 0.0025 g to 0.005 g (concentration of 5% by weight in a standard suspension), as in the reaction batch of Example 1, e.g. a heating rate ΔT=2.99 K/s therefore results.

If one changes the particle concentration in a range of 1 to 10% by weight, this leads to a variation of the heating rate of 0.6 to 6.0 K/s.

These heating rates are well suited to the heating and temperature control of a typical PCR reaction or another nucleic acid amplification reaction or also a nucleic acid elongation reaction. In the event that other heating rates should be required in certain situations, these can be set e.g. by means of the variation of the number, size and type of the particles as desired.

The meaning of the term “beads”, particularly “magnetic beads”, as used here in the context of the present invention, corresponds to the conventional understanding of the person skilled in the art in this field. Beads are generally carrier particles, typically in a size range of approximately 100 nm to approximately 10 μm, preferably approximately 500 nm to 5 μm, which can or cannot be functionalized, are preferably functionalized, are often of approximately spherical shape, and in the case of magnetic beads generally comprise smaller magnetic nanoparticles, which are e.g. embedded in a matrix and/or provided with a coating.

These magnetic nanoparticles typically have a size in the range of approximately 10 nm to approximately 900 nm, preferably approximately 20 nm to 500 nm, and can have a broad spectrum of compositions, as listed in more detail in the following, particularly for use in magnetic beads. These magnetic nanoparticles can be used as such for the thermal control according to the invention of enzymatic reactions or as a constituent of larger magnetic beads, as described in more detail in the following. A mixture of different magnetic nanoparticles or a mixture of magnetic nanoparticles with magnetic beads can also be used if appropriate.

In principle, all beads described in the literature and/or available commercially can be considered as magnetic beads for use in the method according to the invention. Suitable beads can be chosen from the broad spectrum of commercially available beads, depending on the desired or required Curie temperature and, if appropriate, desired further surface characteristics and functionalities or produced in accordance with known methods.

The various magnetic beads which are described in the specialist literature (F. Schüth, et al., Angew. Chem. 2007, 119, 1242-1266) or commercially available (http://www.magneticmicrosphere.com) are based on particles made up of pure metals such as Fe and Co, alloys such as CoPt₃, CoPt, FePt etc., or oxidic phases such as γ-Fe₂O₃, FeO, NiO and in particular the spinels Fe₃O₄ or generally M^{II}M^{I1I}₂O₄ (M=Fe, Ni, Co, Mn, Cr, Mg, Zn etc.). For most commercially available magnetic beads, the pure metals and particularly the various iron oxides are preferred.

Various ways are available for producing the respective particles, wherein here in particular co-precipitation, thermal decomposition and/or reduction of precursor molecules, synthesis in micelles and hydrothermal synthesis are preferred.

Co-precipitation is a very simple method to obtain in particular iron oxides (e.g.: Fe₃O4, γ-Fe₂O₃) from aqueous Fe²⁺/Fe³⁺ solutions (chlorides, nitrates, sulphates, etc.) and bases (alkali hydroxides, ammonia solution, organic amines, etc.) in high yield and relatively narrow particle size distribution. The reaction conditions (Fe²⁺/Fe³⁺ ratio, pH value and ionic strength of the medium, type of salts, temperature: 20-90° C., inert gas atmosphere) also determine the type, quality and size of the particles and must accordingly be reproduced exactly.

Due to the thermal decomposition of metallorganic precursors (metal carbonyls, metal acetylacetonates or metal cupferronates(N-nitrosophenylhydroxylamine complexes) in high-boiling organic solvents (petroleum ether, toluene, long-chain ethers, etc.) which contain surfactants (fatty acids, oleic acid, hexadecylamines), one obtains good yields of very narrowly distributed (monodisperse) particles of high quality in a reaction which is not very easy to control (100-320° C., inert gas atmosphere, nucleation in a short space of time—growth over hours and days). In a reducing atmosphere (H₂), nanoparticles of the pure metals or alloys (Fe, Co, FePt, CoPt₃,) can be obtained in this manner, whilst oxidizing conditions (air, O₂, (CH₃)₃NO) make metal oxides (Fe₃O₄, Co₃O₄, MnO, NiO, Cr₂O₃) available. Relatively small (2-20 nm) particles are however generated for the most part due to the long growth period.

In the (inverse) micelles of microemulsions, predominantly the various spinels are obtained, the size (1-50 nm, narrow distribution) and shape of which depends on the ratio of water and “oil phase” (in particular toluene), but also on the stabilizing surfactants. The micelles in this case constitute a nanoreactor for the precipitation of metal salt solutions by means of bases. Such batches generally provide only a low yield, however, and can only be scaled upwards with difficulty, as larger and larger quantities of organic solvents are required compared with the aqueous portion.

Magnetic beads for use in the method according to the invention preferably have magnetic particles, generally nanoparticles, the composition of which is selected from the group which consists of metals, metal oxides and metal alloys of Fe, Ni, Co, Cu, Mn, Cr, Mg, Zn, La and Sr. Depending on the application, particles of another composition (e.g. as disclosed in the prior art mentioned above) may be suitable, however.

The magnetic beads or magnetic nanoparticles used in the method according to the invention further preferably comprise particles with a settable Curie temperature. Some non-limiting examples of suitable compositions of the magnetic particles are a Cu/Ni alloy, La₁₋ₓSrₓMnO₃ (0≦x≦1), A₁₋ₓZnₓ+Fe₂O₄ (A=Ni, Mn or Cu; 0≦x≦1) and AₓB_{y}+Fe₂₊ₓO₄ (A, B=Ni, Mn, Mg, Ca or Cu; x+y+z=1) (cf. Table 1).

The Curie temperature at which the specific power absorption rate changes can thus be predetermined by means of the selection of the alloy of the magnetic particles. This can for example be used for setting defined temperature ranges which should not be exceeded in the reaction to be controlled during the heating procedure with alternating magnetic fields.

TABLE 1
Magnetic alloys with settable Curie temperature

| Patent | | | | | |
|---|---|---|---|---|---|
| number/public | Title | Holder/author | Brief description | Alloy described | |
| U. S. 6731111 B2 | Validity | Toshiba | Magnetic powder for | A₁—ₓZnx + Fe₂O₄ | the larger the proportion |
| | determination using | | producing magnetic ink | (A = Ni/Mn/Cu) | of NiZn is, the smaller is |
| | magnetic ink having | | with Curie | | the Curie temp. |
| | magnetic powders | | temperatures between- | | |
| | with different curie | | 50° C. and 150° C. and a | | |
| | temperatures | | particle size <10 μm. | | |
| D. E. 4103263A1 | Fine particulate | BASF | Method for the | A_{X}B_{Y} + Fe_{2 + z}O₄ | X + Y + Z = 1 |
| | low coercive ferrite | | production and | (A/B = Ni/Mn/Mg/Ca/Cu) | Curie temp. between |
| | and method for its | | also use for the | | 18° C. and 200° C. ; |
| | production | | absorption of | | particle size <0.5 |
| | | | electromagnetic | | lam |
| | | | radiation | | |
| J Biomed Mater Res | T(C)-tuned | Prasad NK, | Magnetic particles | La₁₋ₓSrₓMnO₃ | Curie temp. below 70° C. ; |
| B Appl Biomater. | biocompatible | Rathinasamy K, | for the hyperthermic | | particle size 20-100 nm |
| 2007 Oct. 5 | suspension of | Panda D, | treatment of cancer | | |
| | La(0.73)Sr(0.27)MnO(3) | Bahadur D. | | | |
| | for magnetic | | | | |
| | hyperthermia. | | | | |
| Biomagn Res | Physically synthesized | Martin Bettge, | Magnetic particles | Cu/Ni alloy | 33 % Cu and 67% Ni |
| Technol. 2004 | Ni—Cu nanoparticles for | Jhunu Chatterjee | for the hyperthermic | | corresponds to a Curie |
| May 8; 2(1):4 | magnetic hyperthermia | and Yousef Haik | treatment of cancer | | temp. of 0° C.; 29% Cu |
| | | | | | and 71% Ni corresponds |
| | | | | | to a Curie temp. of |
| | | | | | 41-46° C. |

The magnetic beads used in the method according to the invention can have the magnetic particles embedded in a natural or synthetic polymer matrix.

Beads of this type can e.g. be obtained by means of the embedding of the magnetic particles in natural (e.g. polysaccharides such as dextran, sepharose, polypeptides such as poly-L-aspartate, poly-L-glutamate, polylactides such as poly-P, L-lactide) or synthetic polymer matrices (e.g. polyvinyl alcohol, polystyrene (derivatives), poly(meth)acrylates and -acrylamides, polypyrroles, polyesters, poly-s-caprolactam, etc. and copolymers also with natural polymers) or by means of inorganic coatings (e.g. SiO₂, Au, carbon).

In the case of the encapsulation of magnetic particles, either small particles (e.g. of ferrofluids) distributed homogeneously in the carrier matrix or larger ones in the form of core shell particles can be built up. A further option consists in the infiltration of (organic/inorganic) porous materials by means of very small magnetic nanoparticles or solutions of Fe²⁺ and other metal ions (Fe³⁺, Co³⁺, Ni²⁺, Mn²⁺, etc.) and the subsequent formation of the magnetic particles (e.g. ferrites) in the matrix. Also such beads which have the magnetic particles embedded in an inorganic or organic porous carrier matrix can be used advantageously in the method of the present invention.

Particularly in the case of matrix-dispersed particles (“polymer beads”), the size of the beads (up to 5 or 10 μm) for the most part has nothing to do with the size of the magnetic particles contained (often only a few nm), which can be confirmed easily by means of the measurement of the magnetization curve (small particles show a narrow hysteresis).

Both polymers and SiO₂-coated magnetic particles are suitable for being equipped with different functionalities. Functionalized chloro- or alkoxysilanes can be bound to the SiO₂-coating. In this manner, polymerization initiators (e.g. for ATRP) can also be coupled to the particles in order to generate typical core-shell particles with a magnetic core and a polymer shell. These magnetic beads (for the most part polymer particles with iron oxide particles polymerized in or iron oxide particles with a silica coating) can be sourced from manufacturers such as for example Bangs Laboratories Inc., bioMerieux GmbH, Chemagen Biopolymer Technology, Chemicell GmbH, Invitrogen (Dynal), MagnaMedics GmbH, Merck KGaA/EMD Chemicals, Promega, Qiagen, etc. The sizes lie in the range of a few ten nanometers up to approximately 3 μm and a magnetite content between 10 up to >90%.

Such coated and, if appropriate, functionalized beads are particularly preferred for use in the present invention.

Depending on structure, content of magnetizable particles (overall magnetizability) and functionalization, these beads are suitable for various applications. The important conventional applications in the field of life sciences and diagnostics lie in the manual and automated purification of nucleic acids, the affinity purification of recombinant proteins or other biomolecules and cell separation with antibody-coated magnetic beads. The base particles with, for example carboxy- or amino-functionalities can be used for user-specific covalent immobilizations of ligands (e.g. streptavidin, protein A, antibodies, lectins, enzymes such as trypsin, benzonase). In this manner, it is possible to immobilize single-stranded nucleic acids such as for example PCR products, synthesized oligonucleotides, cDNA or RNA on the bead surface.

In the case of an “elongation on the bead” by means of polymerases in accordance with an embodiment of the method according to the invention, an orientation of the single-stranded nucleic acids 5′ to 3′ is required (5′ end immobilized on the bead, whilst the 3′ end remains free). The use of commercially available kits lends itself to this, which kits enable the user-friendly functionalization of surfaces of magnetic beads. This for example includes the ULS^{1M} system (Universal Linkage System) of the company Biozym Scientific GmbH and various linker systems (such as amino-, thiol-, phosphate-, biotin- and spacer C3 linker systems), as these are e.g. offered by the company biomers.net GmbH.

The method according to the invention brings about a temperature control with the use of external alternating magnetic fields in a nucleic acid amplification reaction, such as e.g. a PCR or also other biological and chemical reactions, which require temperature regulation, in a closed chamber. It thereby becomes possible to control and to influence reactions without direct heat coupling (e.g. with heating or peltier elements). The advantages in particular lie in the contactless magnetic control of heating procedures within a closed reaction system, such as for example within a diagnostic cartridge. The same magnets can be used for moving magnetic nanoparticles (beads) as carriers for target molecules, for example in the case of a cell separation, sample preparation/processing and detection of the target molecules. This contributes significantly to the simplicity and compactness of a diagnostic measurement system. Further, the contamination risk due to a direct contacting of the cartridge with conventional methods, due to the spreading of substances and the mechanical wear of components in the analysis system is reduced.

Some modern analytical systems are laid out in such a manner that, following a PCR on the bead, the detection of the denatured DNA single strand also is effected by means of the magnetic bead carrier located thereon, which then functions as a marker. This detection could for example be effected by means of magnetoresistive sensor elements on a microchip (biochip), on which capture molecules complementary to the single strand are coupled. Here, the bead, which is generally paramagnetic, generates a stray field under the influence of an external magnetic field, which stray field can be detected in the vicinity of the magnetoresistive measurement field. The change of the resistance of the magnetoresistive measurement element (GMR or TMR sensor) is here proportional to the number of the magnetic beads bound on the sensor element. This enables a quantification of the DNA molecules located on the bead.

The amplification of the nucleic acids to be detected, which is required in most cases, generally takes place by means of a PCR reaction which was hitherto controlled in a conventional manner by means of a thermocouple (peltier element) or hot air (or microwaves) with the disadvantages listed above. The advantage of the method according to the invention in particular consists then in the fact that all important steps, namely sample preparation/processing, amplification (PCR) of the target nucleic acids on the bead and the subsequent detection on a cartridge can be controlled in an integrated and contactless manner by means of external magnetic fields (permanent magnets or electromagnets).

The method according to the invention is fundamentally suitable for all chemical or biological enzymatic reactions which need a temperature control. This applies in particular for diagnostic systems which are used for the preparation/processing of a sample, the isolation of target structures or molecules, the amplification of target molecules and the detection of target molecules. In addition to the known PCR used most often in molecular biology as an amplification method of nucleic acids, routinely used alternative methods are for example “strand displacement amplification”, “ligase chain reaction”, “rolling circle amplification”, “nucleic acid sequence based amplification”, “branched DNA”, “transcription mediated amplification”, “hybrid capture” and “invader”. Current methods for the detection of the amplified sequences include the use of fluorescent markers, enzymes, radioisotopes and also magnetic particles.

Furthermore, other enzymatic reactions, such as nucleic acid synthesis and nucleic acid elongation reactions, restriction enzyme digest, the conversion of substrates, but also hybridization and binding interactions (biochips, immunoassays) can be controlled with the method according to the invention as well.

A second aspect of the present invention comprises a reactor, which is suitable for carrying out the method according to the invention, comprising at least one device for generating an alternating magnetic field (1), e.g. a coil, and a reaction space (2) which comprises the reactants and the magnetic beads (21). This reactor can furthermore comprise further components, e.g. a measurement and control device (3) and/or a cooling device (4).

In a more specific embodiment, the reactor is a thermocycler for PCR or another nucleic acid amplification reaction.

A more generalized aspect of the invention relates to an in vitro testing system which comprises a reactor of this type. The in vitro system or the reactor can comprise a diagnostic unit (cartridge) as a constituent, which can contain further components, e.g. for analysis and evaluation, and can also be constructed as an exchangeable unit for single use.

A diagnostic cartridge of this type generally consists of a plastic material, into which microfluidic channels and reaction chambers are incorporated, in which microfluidic channels and reaction chambers chemical, biological and physical reactions, such as for example sample preparation/processsing, target molecule isolation and purification, the amplification of the target molecules (in the case of nucleic acids) and the detection thereof can take place by means of binding to a microarray. These diagnostic cartridges are for the most part used as a disposable part for sample analysis and controlled by means of a device, into which these are inserted. The production takes place with the aid of rapid prototyping, punching, milling or injection molding methods. Examples of diagnostic cartridges, which are generally also designated as lab on a chip systems, are described in patent publications such as DE 102005029809A1 or DE 102005049976A1.

### DESCRIPTION OF THE FIGURES

FIG. 1 shows a schematic illustration of the process of a PCR reaction with bead-coupled primers.

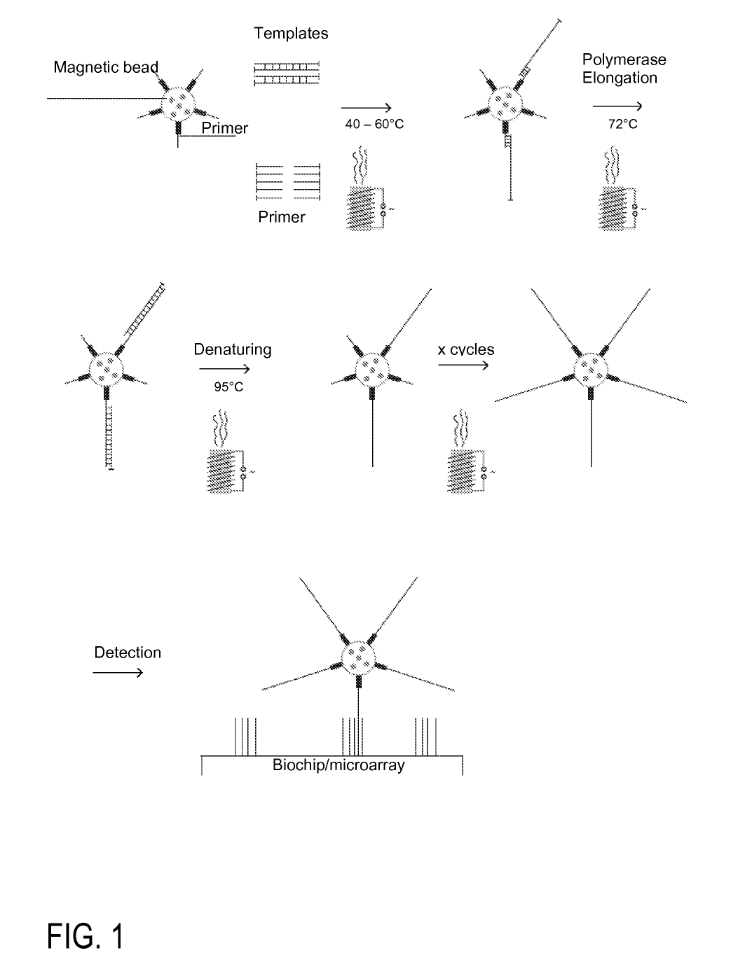

FIG. 2 schematically shows two variants of a reactor according to the invention for carrying out the temperature control by means of the heating of the magnetic beads. Field A in this case shows only the essential components for execution, independently of further constituents, whilst Field B shows a variant in which the reaction space is a constituent of a diagnostic unit (cartridge).

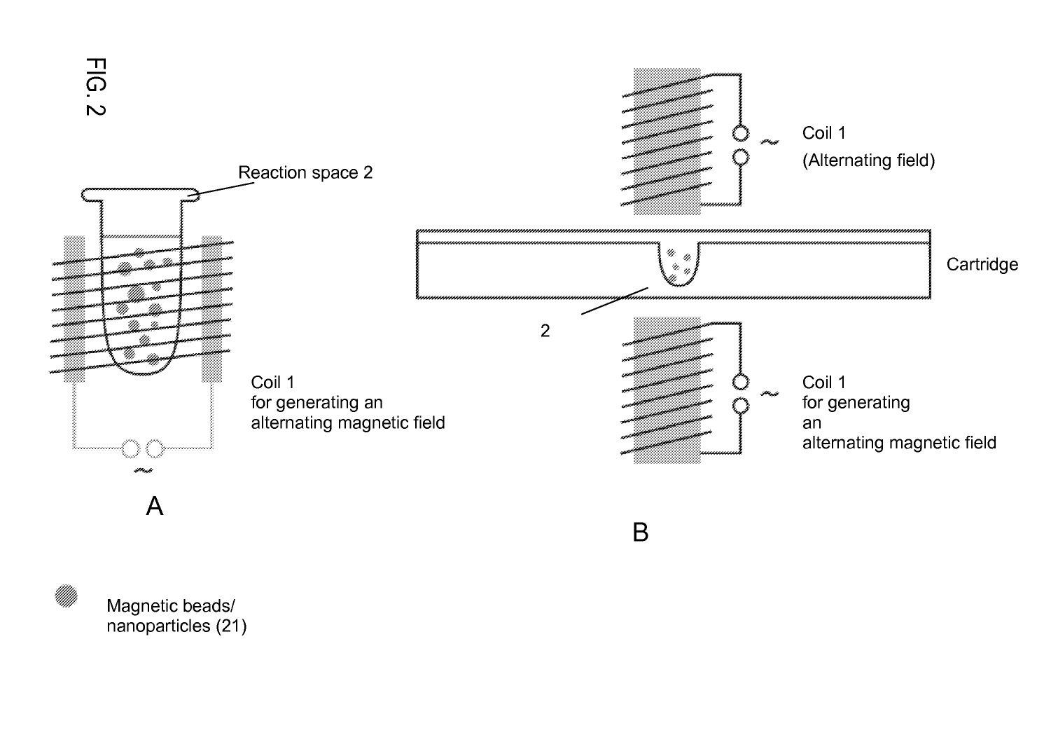

The following exemplary embodiment should explain the invention in more detail, but in no way limit it to the experimental details disclosed there.

### EXAMPLE 1

A typical batch for a PCR reaction comprises:

1.0 μl aqueous solution with DNA template (approximately 100 ng/μl)
2.0 μl per primer (sense/antisense) (10 μM)
1.0 μl Pfu, Taq or another thermostable polymerase (1-5 U/μl)
1.0 μl deoxynucleotides (dNTPs) (dATP, dGTP, dCTP, dTTP per 10 mM)
5.0 μl 10-times concentrated polymerase buffer solution
40.0 μl distilled water
50.0 μl total volume

For generating the alternating magnetic field, e.g. a field strength of 1.6 kAm⁻¹ in the kHz range, e.g. 300 kHz is chosen.

Under such conditions, the specific power loss of magnetite particles (Fe₃O₄) with 25 nm diameter is approx. 250 kJ/kg s.

In the case of the application of the equation specified at the top of page 7, for a water content of 50 μl (0.05 g) and a particle quantity of 0.0025 g (concentration of 5% by weight in a standard suspension), a heating rate ΔT=2.99 K/s results in the above batch.

This heating rate corresponds to the usual heating rates which are achieved with conventional heating elements for PCR reactions of this type and enables the thermal control of the PCR reaction above.

Typical temperatures for the temperature cycles of a PCR reaction are approx. 95-100° C. for the denaturing, 40-65° C. for the hybridization, 70-80° C. for the elongation (polymerase-dependent, e.g. preferably 72° C. for Taq polymerase). Such temperatures can be readily achieved with the method according to the invention.

1. A method for thermally controlling at least one temperature-dependent enzymatic reaction in a presence of magnetic particle or magnetic beads in vitro, said method comprising:
heating of the magnetic beads or magnetic particles to at least one certain target temperature by application of alternating magnetic fields, wherein the temperature-dependent enzymatic reaction is a PCR reaction or another reaction for elongating or amplifiying nucleic acids, including DNA, RNA or hybrids or derivatives thereof, and wherein the beads are functionalized magnetic beads and the reaction takes place directly on the beads.

2. The method according to claim 1, wherein a functionalization of the beads comprises the binding of primer sequences.

3. The method according to claim 1, the method comprises a plurality of cycles of heating and cooling.

4. The method according to claim 1, wherein the temperature-dependent enzymatic reaction is a PCR on the bead reaction.

5. The method according to claim 1, wherein the alternating magnetic fields have a frequency in a frequency range of 1 to 1000, particularly 50-500 kHz.

6. The method according to claim 1, wherein the magnetic beads or magnetic particles comprise particles with a settable Curie temperature.

7. The method according to claim 6, wherein the Curie temperature of the particles with a settable Curie temperature is determined by selecting a composition of the particles in such a manner that a defined temperature range is set, which should not be exceeded in the reaction to be controlled during the heating step.

8. The method according to claim 1, wherein the magnetic beads or magnetic particles comprise magnetic particles comprising at least one member, selected from the group consisting of Fe, Ni, Co, Cu, Mn, Cr, Mg, Zn, La, and Sr, metal oxides thereof and metal alloys thereof.

9. The method according to claim 8, wherein the magnetic particles comprise a Cu/Ni alloy, La₁₋ₓSrₓMnO₃ (0≦x≦1), A₁₋ₓZnₓ+Fe₂O₄ (A=Ni, Mn or Cu; 0≦x≦1) or AₓB_{y}+Fe_{2+z}O₄ (A, B=Ni, Mn, Mg, Ca or Cu; x+y+z=1).

10. The method according to claim 1, characterized in that the magnetic beads have the magnetic particles embedded in a natural or synthetic polymer matrix.

11. The method according to claim 1, wherein the magnetic beads have the magnetic particles embedded in an inorganic or organic porous carrier matrix.

12. The method according to claim 1, wherein the magnetic beads comprise magnetic particles which have a silica coating or another coating.

13. The method according to claim 1, wherein the magnetic particles have a size in a range of 10 nm to 500 nm and the magnetic beads have a size in a range of 500 nm to 5 μm.

14. A reactor which is suitable for carrying out the method according to claim 1, comprising a device for generating an alternating magnetic field and a reaction space which comprises reactants and the functionalized magnetic beads.

15. The reactor according to claim 14, which further comprises a measurement and control device and, optionally, a cooling device.

16. The reactor according to claim 14, wherein the reactor is a thermocycler for PCR or an analogous device for another nucleic acid amplification reaction.

17. The reactor according to claim 14, further comprising a diagnostic cartridge.

18. In vitro testing system, comprising a reactor according to claim 14.

19. The in vitro testing system according to claim 18, further comprising a diagnostic cartridge.

20. A method of performing analytical and diagnostic testing, said method comprising using the reactor according to claim 14 to analyze a sample and diagnose a condition.

21. The method according to claim 20, further comprising preparation, processing and/or purification of a sample, isolating target structures and/or target molecules and amplifying and/or identifying target molecules.

22. A method of using of functionalized magnetic beads for thermally controlling a PCR reaction or primer elongation on a bead.
